(19) United States
(12) Patent Application Publication
(10) Pub. No.: US 2010/0068750 A1
(43) Pub. Date: Mar. 18, 2010

(54) METHOD FOR FLUOROMETRICALLY DETERMINING PHOTOSYNTHESIS PARAMETERS OF PHOTOAUTOTROPIC ORGANISMS, DEVICE FOR CARRYING OUT SAID METHOD AND A MEASUREMENT CHAMBER

(76) Inventors: Sergey Iosifovich Pogosjan, Moscow (RU); Yury Valerievich Kasimirko, Moscow (RU); Dmitry Nikolaevich Matorin, Moscow (RU); Galina Yurjevna Risnitchenko, Moscow (RU); Andrey Borisovich Rubin, Moscow (RU)

Correspondence Address: LADAS & PARRY LLP, 26 WEST 61ST STREET, NEW YORK, NY, 10023, US

(21) Appl. No.: 12/440,651

(22) PCT Filed: Sep. 11, 2007
(86) PCT No.: PCT/RU2007/000482

§ 371 Date: Aug. 18, 2009

(30) Foreign Application Priority Data
Sep. 13, 2006 (RU) 2006132691

(51) Int. Cl. C12Q 1/02; C12M 1/34

(52) U.S. Cl. 435/29; 435/286.1; 435/288.7

(57) ABSTRACT

The invention can be used in biology and for environmental studies using fluorimeters. The inventive method consists in producing exiting light pulses having equal amplitude and modifiable duration, in measuring fluorescent chlorophyll characteristics at a constant background illumination simulating the irradiation intensity of an object during studies in the natural conditions and after the adaption thereof in the dark and in determining the state of a photosynthetic apparatus according to the entirety of fluorescent intensity values. The inventive device comprises the even number of measuring light sources, a current stabiliser of the light sources, the outputs of which are connected to electric inputs of the light sources, the input of which is connected to a control unit and a natural irradiation sensor is connected to the current stabiliser of the light sources through said control unit. A measurement chamber comprises the even number of light sources which are arranged by pairs diametrically oppositely to each other in one plane which is perpendicular to the axis of the body.

### FIELD OF THE INVENTION

The invention relates to a field of biology and can be used in environmental studies, in particular, limnology and oceanology for studying and evaluating the state of aquatic medium for measuring the concentration of algae and photosynthesis thereof, as well as in any other field of science, technology and environmental protection where continuous analysis of aquatic medium is required to be carried out using fluorometers.

### BACKGROUND OF THE INVENTION

It is known that under effect of different ecological factors and anthropogenic pollutions on land and aquatic ecosystems the concentration and photosynthetic activity of cells of photoautotropic organisms primarily change. Their changes result in changes in all the remaining ecosystem links. In view of this functioning the photosynthetic apparatus (PSA) is turned to be the most essential for determining the state of a plant. So, recording the photosynthetic characteristics of phytoplankton is a method for evaluating the state of aquatic medium as a whole.

The model for primary photosynthesis reactions currently accepted covers two photosystems PS1 and PS2. PS2 oxidizes water separating oxygen and protons and reduces the primary and secondary quinone acceptors Ga and Gb. PS1 transfers an electron of the plastoquinone (PQ) pool to the final electron acceptor Co₂.

The reaction center of PC2 (RC) consists of a special chlorophyll molecule P680 which in an exciting state is a primary electron donor for quinine acceptor Ga. The energy of a light quantum absorbed in PS2 can be transformed into the energy of separated charges P680⁺Qa⁻ which is used in further photosynthetic reactions or lost by emitting a fluorescence quantum or scattering in heat. These three processes are characterized by velocity constants Kₚₕ, Kᵣ and K_{d}, respectively.

The starting state in which P680 is reduced, and Qa is oxidized is called open. The state formed immediately after the charge separation in the primary pair P680⁺Qa⁻ is called a closed state of RC. In this state a new portion of excitation cannot be used by such a center until it returns to the starting open state by reducing P680⁺Qa⁻ from the secondary donors and oxidizing the primary acceptor with the secondary electron acceptors.

In the open state of RC the efficiency of using the excitation energy in photosynthesis is high, a probability to lose the energy is minimum, the fluorescence quantum efficiency equal to the ratio of K_{f}/(Kₚₕ+K_{f}+K_{d}) is minimum and is about 2%. In the case of constant intensity of exciting light causing no closing RC the fluorescence intensity corresponds to the value F₀. The high chlorophyll fluorescence efficiency by using the light energy in the primary photosynthetic reactions is provided by photochemical chlorophyll fluorescence quenching. In the closed RC photochemical charge separation becomes impossible, the fluorescence quantum efficiency increases to K_{f}/(K_{f}+K_{d}) corresponding to the intensity value Fₘ, that is about 5%. The difference between the maximum and minimum values of fluorescence intensity (Fᵥ+Fₘ₋Fₒ−variable fluorescence) is proportional to the part of light energy which is used in photochemical photosynthetic reactions in open reaction centers of PS2. The intensities ratio of variable and maximum fluorescence Fᵥ/Fₘ (relative fluorescence variable) is equal to the efficiency of using the RC light energy, that is, allows determining the efficiency of light utilization in the process of photosynthesis. This nondimensional energetic characteristic of photosynthesis similar to the efficiency coefficient is universal and undependable on species-related specificity of organism.

On exposure of a dark-adapted plant to exciting light, some change of chlorophyll fluorescence intensity in time (specific induction fluorescence curve) having several phases is observed. First the fluorescence intensity rapidly increases to the level that corresponds to the fluorescence quantum efficiency in open RC (Fₒ). Then, under sufficiently high intensity of acting light, the fluorescence quantum efficiency may reach Fₘ. The further phases of fluorescence chlorophyll induction ultimately results in decreasing the fluorescence efficiency. This effect is caused by increasing the photochemical quenching under accelerating the electron transport and appropriately decreasing the reduction degree of quinine acceptors of PS2, as well as developing the processes of nonphotochemical quenching, which are provided by some processes associated with producing the proton gradient on the photosynthetic membrane.

Thus, measuring different parameters of chlorophyll fluorescence makes it possible to get information on the state of the photosynthetic apparatus of an object:

So, measuring the following parameters of fluorescence:
Fo is the chlorophyll fluorescence intensity value, in the absence of constant illumination after long adaptation of a sample in the dark under excitation with testing light not leading to closing the RC and not resulting in changing the state of the photosynthetic apparatus.
Fm is the chlorophyll fluorescence intensity value in the absence of constant illumination after long adaptation of a sample in the dark under light excitation leading to completely closing RC and reaching the stationary level.
Ft is the chlorophyll fluorescence intensity value under long constant illumination.
F′m is the chlorophyll fluorescence intensity value under long constant illumination of an object and light excitation thereof leading to completely closing RC of the photosynthetic apparatus.
   allows computing such values of the state of photosynthesizing organisms as:

1. The maximum quantum yield of charge separation in PS2 as a ratio of Fv/Fm=(Fm−Fo)/Fm. This parameter is proportional to the portion of the active RC of PS2 in the dark and correspondents to the potential efficiency of photosynthetic processes.

2. Photochemical quenching in background light—qP=(F′m−Ft)/F′m−Fo′),

3. Nonphotochemical quenching in background light—NPQ=(Fm/F′m)−1,

4. The quantum yield of photochemical transformation of absorbed light energy in PS2 as a ratio of Y=(F′m−Ft)/F′m, reflecting the parameters of non-cyclic transport of electrons under photosynthesis,

5. The absolute value of Fo as a abundance index of plankton in water;

6. The rate of electron transfer from Qa to Qb, as well as the size of light antenna and quinone pool which can be calculated by the intensity growth kinetics of chlorophyll fluorescence from Fo to Fm and others.

The fluorescent methods of evaluating the physiological state of plant organisms are the most objective, nondestructive and make it possible for a short time to receive data on the state of PSA objects in the natural habitat on a real time-basis.

Known is a single-beam method of recording fluorescence (Matorin D. N., Venedictov P. S. Chlorophyll luminescence in microalgal cultures and natural populations of phytoplankton//Moscow: Itogi nauki i tekhniki, VINITI. Ser. Biophysics. 1990. V.40. P. 49-100) by illuminating an object with constant light through a shortwave (usually blue) filter followed by recording the induction changes of fluorescence intensity through a crossed red filter protecting a fluorescence detector from exciting light. Inasmuch as the fluorescence efficiency is small, a photomultiplier is used as a detector. Using the same beam of light to excite fluorescence and actuate the photosynthetic processes during which the state of photosynthetic apparatus and fluorescence quantum yield changes does not allow to uniquely interpret the results of such measurements.

Known is a double-beam for fluorescence recording (Lyadsky V. V., Gorbunov M. A., Venediktov P. S. Pulse fluorometer for studying primary photochemical processes of green plants//Scientific reports of higher school. Biological sciences. 1987, V. 11, P. 31-36.), in which the change of the state of PSA is carried out by a constant light flux, and the excitation of fluorescence in order to probe the changes of PSA is carried out by faint modulated light and the state PSA is evaluated by the changes of fluorescence quantum yield.

For recording a fluorescence modulated signal this method involves a resonant amplifier that does not let pass the constant fluorescence signal excited by the acting light.

This method has a rather narrow dynamic range of intensities of measuring and acting light. So, a rise of saturation intensities of acting light requires rather intensive probing light such that a variable fluorescence signal is higher than the noises of a constant fluorescence signal induced by constant acting light. However, in this case probing light may induce a notable electron stream resulting in an error in the determination of the value Fₒ. Moreover, the known method implies using inhibitors (diuron) making measuring much more difficult, and eventually, is unrealizable in a submerged or flowing variant.

Known is also a method of recording fluorescence by modulating probing light (fluorometer with pulse amplitude modulation (Qunis A., Evain S., Flexas J., Tosti S., Moya I. Adaptation of a PAM-fluorometers for remote sensing of chlorophyll fluorescence//Photosynth. Res. 2001. V. 68. No. 2, P. 113-120) in which as a source of measuring light a light-emitting diode generating very short pulses (1 μs) of red light (650 nm) with a high pulse ratio (the interval between pulses is about 1000 μs). An pulse fluorescence signal is detected by a light-emitting diode and enhanced by a synchronous detector pulse amplifier. This method allows increasing the ratio of the intensities of acting and probing light up to 10⁶ and reliably registering the fluorescence level Fₒ in the intensity wide range of acting light. At the same time, the known method implies as acting light to use constant light which induces multiple actuation of photosynthetic reaction centers (multiple turnover). This hinders interpreting the data, inasmuch as in a steady state the fluorescence frequency is influenced by a lot of factors, the contribution of each factor it is difficult to determine finally leading to a possibility of a controversial evaluation of the results.

Known also is a method of fluorometric determining the photosynthesis parameters of photoautotropic organisms for measuring the parameters of chlorophyll fluorescence (U.S. Pat. No. 4,942,303 IPC G 01 N 21/64, pub. on 1990) based on the “pump-and-probe” method, in which a sample is successively illuminated by three light flashes: “faint probing—powerful saturating—faint probing”. The powerful pump flash induces a single turnover of photosynthetic RC, and the faint probing flash fed in different time after pump serves for determining the kinetics of changing the quantum yield of chlorophyll fluorescence in the process of transition of RC from a closed state into an open state.

Measuring the fluorescence efficiency before and after the saturated flash in the presence of constant acting light of the known intensity makes measuring the photosynthesis rate possible. The pump-and-probe method allows somewhat expanding the range of determined PSA parameters. At the same time, for measuring the absorption cross-section and electron stream rate from PS2 to PS1, the sequence of flashes “probe-pump-probe” should be repeated 30 times, the pump flash intensity being measured from zero to the saturation level, or dead time between pump and the second probing flash is changed from 80 μs to 300 μs. Realizing these two experimental protocols takes 5 minutes to 10 minutes of fluometer operation to perform appropriate measurements. This confines the scope of use of the known method, for example, for obtaining a vertical profile of phytoplankton fluorescence in the ocean where these protocols are often required to be performed every other meter of water, said time becomes inadmissibly much. The need to maintain the intensity of the probing flash lower than 1% of the saturation level of PS2 results in a low signal/noise ratio, particularly at the low concentration of chlorophyll allowing no gaining appropriate measuring sensitivity.

The known pump-and-probe fluorometer implies using two separate excitation channels (two flashes) expanding a construction and increasing the fluorometer cost. Besides, the completely performing the experimental protocol according to the known method, in particular, for studying phytoplankton in the ocean requires a great amount of electric power. These requirements confine a possibility of long autonomous measuring (on a floating buoy), wherein electric butteries are used for power supply of the fluorometer.

With regard to the assigned objective a set of the parameters of the photosynthetic apparatus being measured are determined and one or other method of excitation and recording fluorescence is used.

The closest of the known technical solutions to the invention described is a method of fluorometrically determining the photosynthesis parameters of photoautotropic organisms (U.S. Pat. No. 5,426,306, IPC G 01 N 21/64, pub. in 1995) comprising light exposure of a sample piece of the medium analyzed to exciting light pulses with the energy sufficient to excite chlorophyll fluorescence in the sample followed by measuring the fluorescence intensity according to which the photosynthetic parameters of the investigated object are determined.

The known method uses as exciting light pulses fast repetition flashes (fast repetition rate, RFF) with adjustable energy and high frequency for gradual and increasing saturation of PS2 in phytoplankton. The known method allows fast getting data of the functional value of absorption cross-section of RC, energy transfer the between photosynthetic units of PS2, photochemical and nonphotochemical quenching fluorescence and electron transfer kinetics on the acceptor side of PS2. However, the possibilities of the known method of fluorometrically determining the state and activity of PSA of photosynthesizing organisms do not suppose multiple measuring the fluorescence intensity of Fₒ on one sample resulting in great errors in measuring this key parameter. For determining each parameter of the state of PSA the known method uses an individual sample also introducing great errors. The known method does not allow determining different types of quenching and, therefore, calculating the light curve of electron transport and determining the optimum photosynthesis zones. The stability of the antioxidant system also cannot be determined by the known method.

Moreover, the known method does not allow defining the contribution of some species of organisms to production characteristics of the phytoplankton community. Furthermore, the methods of mathematic modeling cannot be practically applied to the known measuring method as each pulse has to be modeled separately and in a series of 100-200 pulses it rather difficult. Therefore, using a mathematic model in the known method to determine (calculate) the constants of electron transfer reactions rates in the process of photosynthesis gives only distantly approximate values or is impossible at all.

The closest one of the known devices for determining the state of photosynthesizing organisms to the invention described is a device realizing the method according to the aforesaid patent comprising a measurement chamber, a source of measuring light capable of exciting the fluorescence of a sample, a device for measuring the fluorescence of a sample and a control unit connected to a computing machine, the measuring light source and device for measuring the fluorescence of a sample.

The construction of the known fluorometer allows carrying out continuous measuring the photosynthetic parameters and photosynthesis rate, both in the dark and in the natural lighting. However, the functional abilities of the known method are confined and do not allow simultaneously measuring a required number of parameters characterizing PSA of organism required to calculate and build a light curve of electron transport or to build mathematic models of PSA according to which the features and PSA parameters directly immeasurable can be determined. Moreover, inasmuch as a small fluorescent signal in the known device is measured on the background of intensive solar irradiation, measuring in an open chamber in the water surface layers under intensive sun light is impossible.

Furthermore, measurement chambers in all the known devices for measuring fluorescence do not provide suppressing a ghost signal from a photoreceiver caused by that a great deal of scattered light falls on exciting light getting to the walls and other constructive elements of the measurement chamber. At the same time, measuring the parameters of phytoplankton fluorescence in the natural conditions characterized by the marginal content of phytoplankton cells requires high sensitivity of a device.

All the aforesaid factors confine a possibility to use the known methods and devices.

### SUMMARY OF THE INVENTION

The present invention is based an objective to increase the objectivity and accuracy of a fluorometric evaluation of the activity of the photosynthetic apparatus of photoautotropic organisms by carrying out multiple measurements with high time resolution on one sample simultaneously determining the contribution of individual species of organisms into the production characteristics of the ecosystem and investigating the abundance of phytoplankton and the in situ functional state thereof, as well as through a possibility of determining the production characteristics thereof in the natural conditions on a real time basis.

Another objective of the present invention is to enlarge the scope of use by implementing a possibility to calculate electron transport light curves and to apply the methods of mathematic modeling and thereby determine additional parameters characterizing photosynthetic processes.

The present invention also solves an objective to create a device for determining the state of photosynthesizing organisms with high operational abilities and operating capacity.

These objectives are solved by that the method of fluorometric determining the photosynthesis parameters of photoautotropic organisms comprising a light exposure of a sample of the analyzed medium to exciting light pulses with energy sufficient to excite chlorophyll fluorescence followed by measuring the fluorescence intensity according to which the photosynthetic parameters of the object investigated are determined, measuring fluorescence on one sample of the medium analyzed, exciting light pulses have equal amplitude, and the duration thereof is subsequently changed in accordance with time of electron transfer in some links of the electron-transport photosynthesis chain, measuring the characteristics of chlorophyll fluorescence both under constant background illumination simulating the irradiation intensity of an object during studies in the natural conditions, and after the adaptation of the sample in the dark, and by the integrity of fluorescent intensity values the parameters of the state of the photosynthetic apparatus are determined.

Moreover, for determining the chlorophyll fluorescence intensity at which exciting light pulses do not influence the state of the photosynthetic apparatus, the duration of pulses is selected to be 1-5 μs with an interval between pulses 50-100 ms.

It is appropriate to evaluate the relative size of a light-harvesting complex of pigments of photosynthetic reaction centers by feeding to the sample of a light pulse with duration of 100-200 μs and measuring the chlorophyll fluorescence intensity at least every 10 μs from the start of the light pulse followed by calculating the increment of the chlorophyll fluorescence intensity during the effect of the pulse.

For evaluating the reducing rate of the components in the acceptor part of PS2 it is appropriate to impact on the object by a series of three groups of light pulses of the same amplitude and duration in each group 1-5 μs, 100-200 μs and 200-1000 ms, respectively, at an average irradiation rate density not less than 3000 J·m⁻²·c⁻¹ and to measure the kinetics of changing the fluorescence intensity under effect of these pulses each separately, the fluorescence intensity in response to the pulse duration of 1-5 μs corresponding to the chlorophyll fluorescence intensity value at which the exciting light pulses do not influence the state of the photosynthetic apparatus, by the angle of elevation of the initial section of the induction curve the changes in fluorescence intensity in response to an exciting light pulse with duration of 100-200 μs the relative size of the light-harvesting complex is determined, and by the angle of elevation of the curve the changes the fluorescence intensity in response to the effect of a light pulse having duration 200-1000 μs the relative size of quinone pool is determined.

The maximum level of chlorophyll fluorescence intensity can be determined by feeding to the sample of light pulse having duration 200-500 ms at the irradiation irradiation rate density 3000 W/m².

It is appropriate to measure the kinetics of changing fluorescence (induction curves) by exposing of a sample to a light pulse 300-1000 ms during the exposure thereof at least every other 1 mc to measure the chlorophyll fluorescence intensity.

According to the results of measuring the fluorescence kinetics a mathematic model of the photosynthetic apparatus can be built, according to said model the quantitative features and constants of electron transfer reactions undetermined experimentally are determined.

Measuring the fluorescence parameters are preferably to be done on one sample of the medium studied by subsequent mode switching of exciting pulses after measuring each parameter, whereby the duration of irradiation in each sequent mode is selected to be longer than in the previous irradiation.

It is appropriate to use the method described to determine photosynthetic characteristics of phytoplankton.

It is preferable the selected sample to use simultaneously in order to determine the contribution of individual species of algae into production characteristics of phytoplankton, as well as heterogeneity of populations of individual cells.

In order to determine the contribution of individual species of algae in the production characteristics of phytoplankton and heterogeneity of populations, from the originally selected sample the second sample of the analyzed medium is selected, concentrated, for example, by filtration of water through nuclear filters, the resulting concentrate is distributed in one layer of cells, for example, in a Nageotte chamber, after that by a visual evaluation the species-related composition of cells of phytoplankton organisms is determined.

It is preferably simultaneously with the evaluation of species belonging of the populations to measure in each cell the fluorescence parameters according to the above described method and to determine the heterogeneity thereof, whereas the distribution of cells in the population is determined by the efficiency of photosynthesis processes, and the abundance of pigments in cells—by the chlorophyll fluorescence intensity value at which exciting light pulses do not influence the state of photosynthetic apparatus in the absence of constant illumination after long adaptation of the sample in the dark.

The assigned objective is solved also by that a device for fluorometrically determining the photosynthesis parameters of photoautotropic organisms comprising a measurement chamber, a light source, optically coupled to the measurement chamber and capable of exciting fluorescence of the sample, a module for measuring the sample fluorescence, and connected thereto a control unit connected to a computer, comprises at least one additional light source so that the number of the optically conjugated to the measurement chamber light sources is even, a current stabilizer of the light sources, the outputs of which are connected to the electric inputs of the light sources, and the input is connected to the control unit, and a natural irradiation sensor connected to the stabilizer of the light sources through the control unit.

Furthermore, the light sources are the same and each of them is used as a source of measuring and/or saturating, and/or acting light.

The module for measuring fluorescence of a sample may be in the form of connected to an autonomous high-voltage power supply of a fluorescence detector, for example, a photomultiplier connected to a recording means, for example, with a personal computer.

Furthermore, a signal processor may comprise at least one amplifier connected to an analog-to-digital converter through a synchronous detector, the output of which is connected to the control unit.

It is appropriate to make the signal processor in the form of four series-connected operational amplifiers, the output of which each is connected to the analog-to-digital converter coupled to the control unit through a relevant synchronous detector.

In the preferred embodiment the device for fluorometrically determining the photosynthesis parameters of photoautotropic organisms may comprise a pump, a collector, the first output of which is connected to the measurement chamber, and the second one—to a system for concentrating the second sample piece of the medium, an additional measurement chamber for measuring the florescent parameters of individual cells, as said chamber it is appropriate to use a Nageotte chamber, a microfluorometric adapter consisting of a luminescent microscope with a fluorometric nozzle and a light-emitting diode source of light connected to the control unit through the current stabilizer of the light sources.

Furthermore, the fluorometric nozzle may be made in the form of a module for measuring fluorescence of a sample.

The assigned objective is also solved by that the measurement chamber comprising a body, a light source and a fluorescence detector arranged in the windows of the body, as well as inlet and outlet fittings capable of feeding into the chamber and removing therefrom, respectively, the sample piece of the studied medium, comprises at least one additional light source arranged diametrically oppositely to the first one capable of absorbing light from the appositely arranged source.

It is preferably for the measurement chamber to contain the even number of light sources more than two, which are arranged by pairs diametrically oppositely to each other in one plane which is perpendicular to the axis of the body, wherein each light source is capable of absorbing the light of the oppositely arranged source.

Moreover, the fluorescence detector may be made in the form of a photomultiplier, the axis of the optical system of which coincides with the axis of the body.

As a result of exposure of the sample to light pulses exciting chlorophyll fluorescence of the same amplitude but different duration, each of which corresponds to the electron transfer time at a definite stage of the electron-transport photosynthesis chain, this invention provides a possibility to get respond fluorescence with parameters characterizing reactions executing particularly at this stage of photosynthesis and thereby to measure on one sample the entirety of the values of the functional state of the photosynthetic apparatus of the objects.

Using the data received by the method in accordance with the present invention, measuring with single pulses on one sample of phytoplankton provides also a possibility of building a sufficiently accurate mathematical model of photosynthesis processes according to which the ratio is estimated for the constants of the reaction rates executing in the photosynthetic apparatus of plankton algae but not measured by the direct methods.

This invention also provides a possibility to determine different types of quenching, and therefore, to calculate the light curve of electron transport and to define the optimum photosynthesis zones by creating in the measuring zone illumination reproducing the intensity of natural lighting in the place of the sample harvesting.

Other objectives and advantages of the present invention will become clear from the following detailed description of the method of fluorometrically determining the photosynthesis parameters of photoautotropic organisms and a device for carrying out thereof, as well as concrete embodiments of this method.

### BRIEF DESCRIPTION OF THE DRAWINGS

The invention is explained with drawings (FIG. 1-5), wherein presented are:

FIG. 1 schematically shows a model structure of priming photosynthesis reactions;

FIG. 2 schematically shows a temporary diagram of measuring fluorescence;

FIG. 3 schematically shows a block scheme of a device for measuring parameters of phytoplankton fluorescence;

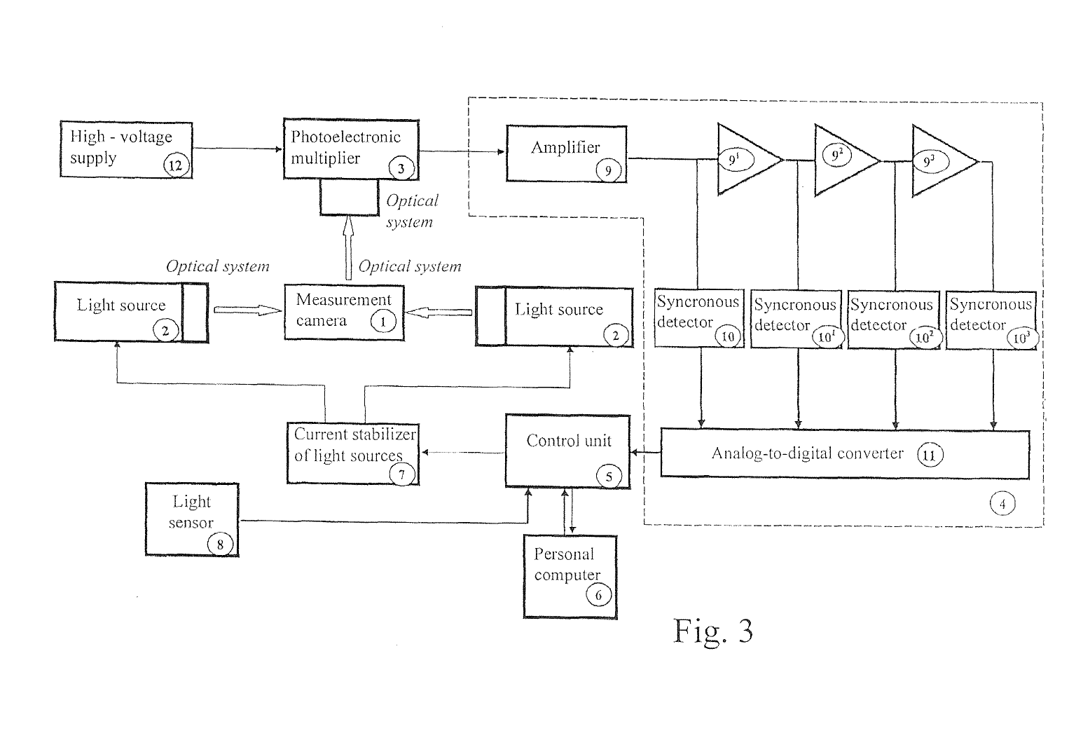

FIG. 4 schematically shows a circuit-design of a measurement chamber;

FIG. 5 schematically shows a physical configuration of an aboard fluorometer as an embodiment of the described invention.

### DETAILED DESCRIPTION OF PREFERRED EMBODIMENTS OF THE INVENTION

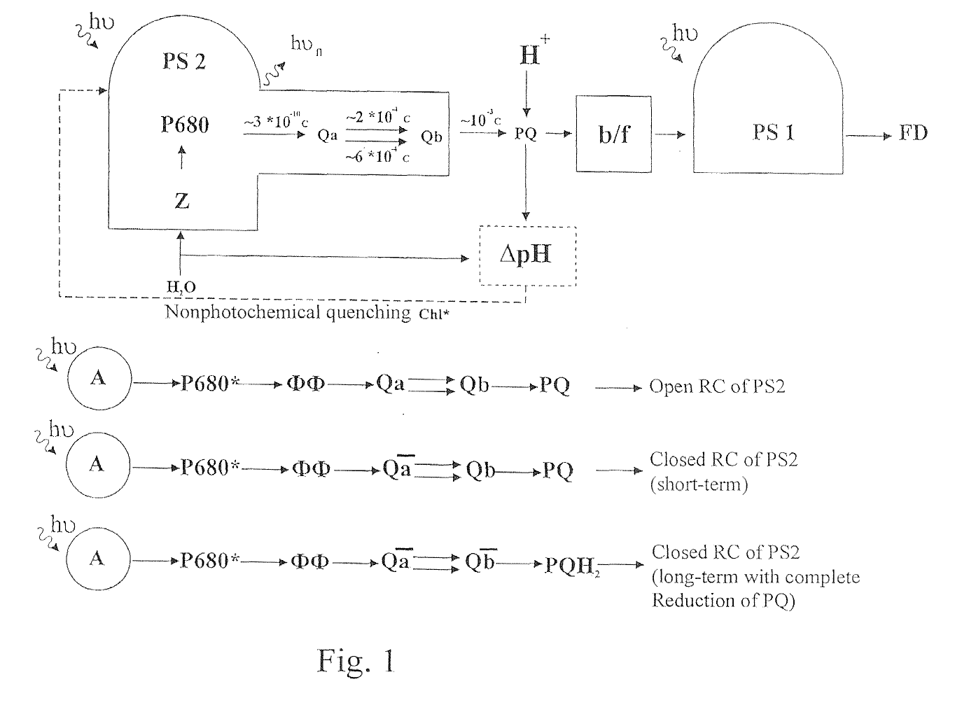

On exposure of the sample to a long light pulse a subsequent actuation of photosynthesis reactions takes place with different executing rates and different products of these reactions (FIG. 1). With that, the chlorophyll fluorescence yield provided by executing these reactions change.

According to the present invention, the sample under study is exposed to pulses of different duration, each of which corresponds to the electron transfer time at a definite stage in the electron-transfer photosynthesis chain resulting in respond fluorescence with parameters characterizing the reactions executing at this particular stage of photosynthesis. So, the characteristic time of one of the first reactions—reducing Qa is about 100 μs, thus, at the duration of a pulse greatly less than 100 μs the actuation of photosynthesis reactions does not practically occur, and that is why under feeding a light pulse of 1-5 μs the level of fluorescence corresponds to the initial level of chlorophyll fluorescence, when all RC are in the “open” state. At the duration of a light pulse of 100-200 ∞s the process of reducing Qa takes place. In doing this, Qa is not practically oxidized, as the following processes of electron transfer through the electron-transport chain happen at greatly less rates. This allows measuring the relative size of the light-harvesting complex by the angle of elevation of the initial section of the changes of the fluorescence intensity. The larger the angle of elevation, the larger the relative size of the light-harvesting antenna.

In response to the effect of exciting light at the duration of 200-1000 ms the full reduction of Qa and Qb and the following pool happens. Thereby the oxidation of the quinone pool in further reactions has greatly less rate. In these conditions the relative size of the quinone pool can be measured by the angle of evaluation of the fluorescence intensity changing curve.

Thus, on exposure of the piece to light pulses exciting chlorophyll fluorescence of the same amplitude but different duration a possibility is gained to measure on one sample some parameters of the functional state of the photosynthetic apparatus of the objects.

In this doing, the measurements of parameters of chlorophyll fluorescence of an object are performed both at the background of acting light equivalent to the natural light in the point of sample harvesting, and (or at the background of the selected value of irradiation intensity) when photosynthesis reactions are actuated and have a fixed-ratio nature, and after adaptation of the sample in the dark.

Creating in the measuring zone illumination reproducing the intensity of natural lighting in the point of sample harvesting allows determining different types of quenching and, therefore, calculating the light curve of electron transport and to find optimum photosynthesis zones.

As increasing the duration of a pulse above 1000 ms, the actuation of a lot of reactions of photosynthesis occurs making it difficult to interpret the resulting data and direct unique measuring parameters without adaptation of the sample in the dark for about 10 minutes becomes impossible. The longer pulse, the longer should be the time of dark adaptation thereafter.

After adaptation in the dark the electron-transport chain of photosynthesis is fully unloaded from electrons and the mechanisms adjusting the primary processes of photosynthesis are off. When light is on, both light and dark reactions adjusting photosynthetic process are actuated. The shorter a light pulse, the less the time in which the following pulse may be fed without changing the photosynthetic apparatus. Therefore, short pulses are fed and Fo is measured first, then longer pulses are fed for measuring the remaining parameters saving time of each measuring cycle. For reliable statistic analysis of the resulting data, several pulses (groups) of the same duration are fed.

The data obtained by measuring with single pulses for one sample of phytoplankton make it possible to build a sufficiently accurate mathematical model of photosynthetic processes according to which the constant ratios of reaction rates executing in the photosynthetic apparatus of plankton algae but immeasurable by the direct methods are calculated.

The method for determining the state of photosynthesizing organisms in accordance with the present invention is shown by a concrete example of investigating the characteristics of chlorophyll fluorescence of phytoplankton cells. Such a choice is grounded by that almost half photosynthetic biological products of the Earth falls on phytoplankton.

Harvesting a water sample containing phytoplankton cells is carried out by any known method with regard to the objectives and conditions of implementing studies.

The water sample is divided into two pieces, one of which is placed into the measurement chamber where illumination is created simulating the irradiation intensity of an object at the moment in the natural conditions and the other piece is concentrated and placed in an individual unit to study individual medium cells and to determine the contribution of individual species of algae in the production characteristics of phytoplankton,

Previously the most informative fluorescent indices are selected for the supposed studies followed by developing the protocol of measuring and the hardware implementation thereof set for the conditions of exciting fluorescence for correctly measuring the fluorescent parameters of the functional PSA state of an object.

The sample is exposed to exciting light pulses with the selected algorithm-modifying duration and chlorophyll fluorescence of phytoplankton cells occurring in response of light exposure is measured. At all the modes of light exposure of the sample the exciting light pulses have the same amplitude.

Below there is given an example of algorithm of measuring simultaneously on one sample to define the entirety of parameters of fluorescence considered above using pulses of three durations.

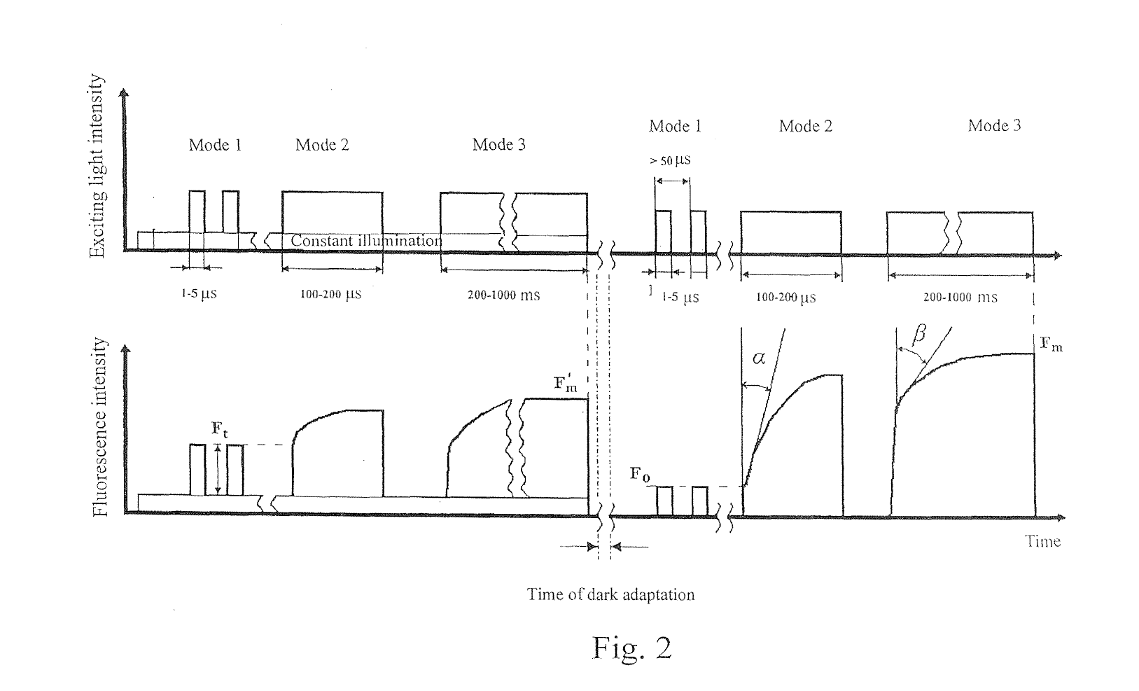

Preliminarily, in the measurement chamber constant illumination equivalent in the number of quanta to the natural background irradiation in the sample harvesting point is created or the irradiation intensity is preset by a operator relying on the mean typical values of the natural irradiation in the sample harvesting point throughout carrying out the first measurement stage in the three following modes (FIG. 2).

1. A mode of determining the chlorophyll fluorescence intensity wherein the pulses of exciting light do not influence the state of the photosynthetic apparatus.

The duration of light pulses is 1 to 5 μs, the interval between pulses is 50 to 100 ms, with the average density of irradiation rate—not greater than 0.4 W·m⁻². In this mode the average value of chlorophyll fluorescence intensity Ft is determined.

The number of measurements is selected relying upon the necessity of achieving the preset value of error of mean, either automatically or in accordance with the operator's calculations.

With the average density of irradiation rate not greater than 0.4 J·m⁻²·s⁻¹ not more than 1% of RC per pulse is closed and the next pulse can be fed already in 10-100 ms, as for this time all RC closed with a short pulse will return to the open state.

After mode 1 is completed mode 2 is turned on.

2. A mode of determining the chlorophyll fluorescence intensity gain during the time of pulse exposure to evaluate the relative size of the light-harvesting complex of pigments of photosynthetic reaction centers.

To make these measurements, a light pulse with duration of 100-200 μs is fed to a sample, every other 10 μs the chlorophyll fluorescence intensity is measured and the average gain of chlorophyll fluorescence intensity as effected by the pulse is calculated as a derivative of time intensity.

The number of measurements is selected relying upon the necessity of achieving the preset value of error of mean, either automatically or in accordance with the operator's calculations.

After mode 2 is completed mode 3 is turned on.

3. A mode of determining the kinetics and steady level of chlorophyll fluorescence intensity with complete saturation with light of the photosynthetic apparatus of phytoplankton cells is produced by a light pulse with duration of 200-1000 ms and the average irradiation rate density of 3000 J·m⁻²·s⁻¹. Chlorophyll fluorescence is measured every 10 μs from the outset of the light pulse. The chlorophyll fluorescence intensity rate therein is inversely proportional to the sizes of the quinone pools.

With all the modes of measuring the chlorophyll fluorescence intensity, pulses of exciting light have the same amplitude. Different values of the average power densities of light exciting chlorophyll fluorescence are obtained by different pulse durations.

4. Then the constant background illumination simulating the light intensity for a sample in the natural conditions is turned off. After the adaptation in the dark of the same water sample piece chlorophyll fluorescence is measured in modes 1, 2 and 3 but without constant background illumination.

In accordance with the findings of measurements with constant illumination and without it fluorescence parameters are determined:
Fo is a chlorophyll fluorescence intensity value in response to pulses of exciting light with duration of 1-5 μs, in the absence of constant background illumination and after the adaptation of the object in the dark.
Fm is a value of maximum level of chlorophyll fluorescence intensity in response to pulses of exciting light with duration of 200-1000 ms, in the absence of constant background illumination and after the adaptation of the object in the dark.
Ft is a value of chlorophyll fluorescence intensity under constant background illumination in response to pulses with duration of 1-5 μs.
F′m is a value of maximum level of chlorophyll fluorescence intensity in response to a pulse of exciting light with duration of 200-1000 ms under constant background illumination.

Of these parameters according to the formulas given before:

1. Maximum quantum yield of charge separation in PS2 as a ratio of Fv/Fm=(Fm−Fo)/Fm. This parameter is proportional to the portion of active RC of PS2 in the dark;

2. Photochemical quenching in background light—qP=(F′m−Ft)/F′m−Fo′);

3. Nonphotochemical quenching in background light—NPQ=(Fm/F′m)−1;

4. The quantum yield of photochemical transformation of absorbed light energy in PS2 with background irradiation as a ratio of Y=(F′m−Ft)/F′m, reflecting non-cyclic transport of electrons under photosynthesis;

5. The absolute value of Fo as a abundance index of plankton in water;

6. The rate of electron transfer from Qa to Qb, as well as the relative size of light-harvesting antenna complex and the relative size of quinone pool which can be calculated by intensity growth kinetics of chlorophyll fluorescence from Fo to Fm.

The resulting measuring data are introduced into a mathematical model of photosynthetic processes and other parameters unmeasured by the direct methods are calculated.

To determine the contribution of individual species of algae into the production characteristics of phytoplankton the second sample piece is tightened by water filtration through nuclear filters, the resulting concentrate is distributed in one layer in a Nageotte chamber with capacity of 70 μl, after that the species composition of cells of phytoplankton organisms is visually determined. On each cell of a definite phytoplankton species being in the chamber the parameters of fluorescence are measured by the method which is applied to measure fluorescence of the first sample, after that the distribution of algal species is determined in accordance with the efficiency of photosynthesis processes and the relative content of pigments in cells (in the size of F₀). Thus, first the specific belonging of the cell under study is determined in the view field of microscope, and then the fluorescent characteristics thereof are measured. These measurements are done on some tens of individual cells of each dominant of phytoplankton and in accordance with the measuring findings the species composition and the number of cells of each phytoplankton algal population is determined, as well as the distribution of cells of each algal species according to the relative content of photosynthetic pigments and efficiency of the primary photosynthetic processes.

For measurements a microfluorometric adapter consisting of a luminescent microscope with a fluorometric nozzle and light-emitting diode light source connected to the feed system of exciting fluorescence light pulses.

The measurements on individual cells allow determining the heterogeneity of populations of single microalgal cells.

The entirety of the fluorescence intensity values of Fo, Fm, Ft, F′m and transition kinetics from Fo to Fm of the aggregate sample piece of phytoplankton and individual cells allows by the known dependencies and on the basis of the mathematic model to determine on one sample the state of the photosynthetic apparatus of the phytoplankton community as a whole and individual species of algae said community comprises.

The device realizing the present invention is configured as follows.

Measurement chamber 1, optically is coupled to light sources 2 and fluorescence detector 3, the output of which through signal processor 4 is connected to control unit 5 connected to data recording and processing unit 6, for example, a computing machine, in particular a personal computer. With that, the inputs of light sources 2 are connected to the outputs of current stabilizer 7 of the light sources connected the input of which is connected to the output of control unit 5 as which a microprocessor can be used. Sensor 8 of acting light is connected to the output of control unit and can be made, for example, in the form of a photodiode with a light filter-correcting system.

Signal processor 4 can comprise, at least, one amplifier 9 connected to analog-digital converter 11 through synchronous detector 10.

In the preferred embodiment signal processor 4 consists of several series-connected operational amplifiers 9, the output of which each through synchronous detector 10 is connected to analog-digital converter (ADC) 11 the output of which is connected to the input of control unit 5 connected to personal computer 6.

Current stabilizer 7 of light sources 2 is used for converting the driving voltage coming from control unit 5 to the current providing necessary irradiation intensity of light sources 2 in accordance with the preset algorithm. Current stabilizer 7 is made in the form of several independent channels, whose number corresponds to the number of light sources 2 and the output of which each is connected to the electrical input of relevant light source 2. Each channel of stabilizer 7 can be made, for example, in the faun of series-connected a field transistor and ballast resistance connected to the operating output of control unit 5.

Fluorescence detector 3 can be made in the form of a photomultiplier connected to autonomous high-voltage power supply 12, for example, a TRACO's module MHV 12-1.5. As photomultiplier 3 can be used, for example, a photoelectric multiplier PEM-79 provided with adjacent light filter KS 18 allowing to record irradiation having a wavelength 680 nm and more.

The computing machine and the control unit have autonomous direct current power supplies or are energized from a multi-channel voltage converter (not shown in the drawing).

Light sources 2 and photomultiplier 3 contain relevant optical systems.

As light sources 2 light-emitting diodes can be used, each of said diodes is capable of fulfilling functions of measuring light and/or saturating light, and/or constant illumination, for example, powerful light-emitting diodes L400CWO12K, T4 Round (Ledtronics, Inc.) with a wavelength with maximum irradiation of 612 nm.

Light sources 2 in the even number are evenly arranged in the windows of measurement chamber 1 around its axis in one plane perpendicular to the axis of the body of the chamber. Moreover, light sources 3 are arranged by pairs diametrically oppositely to each other and each of them is capable of absorbing light from the opposite source.

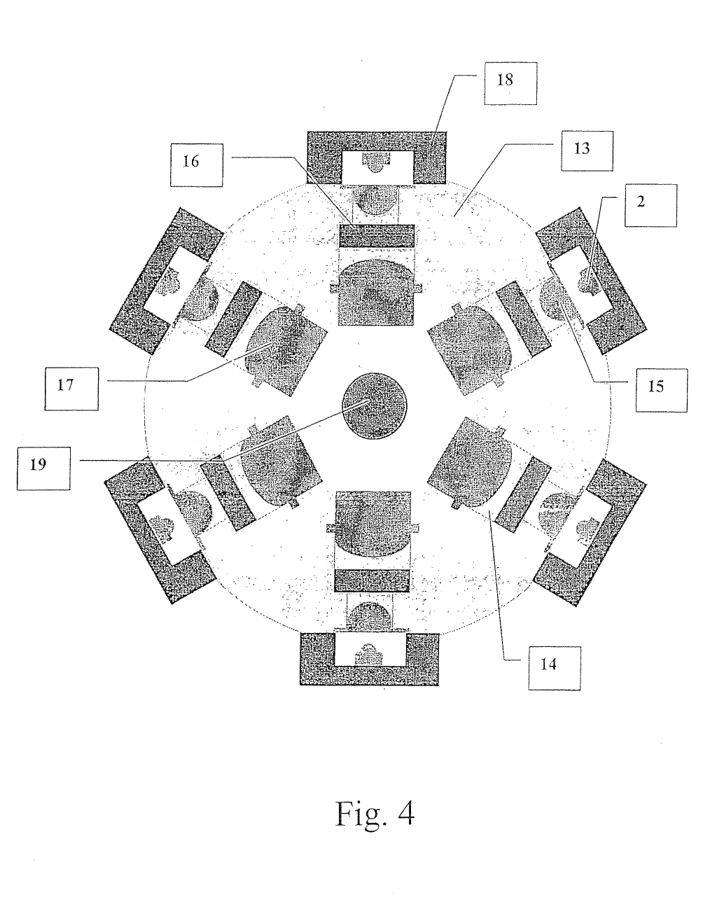

In body 13 of measurement chamber 1 (FIG. 4) there are windows 14, wherein the optical systems of light sources 2 are arranged. Each optical system comprises spherical lens 15, light filter 16 and long-focus lens 17, wherein light source 2 is attached to heat sink 18 conjugated in the area of window 14 to body 13. In window 19 the optical system of photomultiplier 3 is arranged. Windows 14 for light sources 2 are arranged around the axis of body 13 capable of arranging light sources by pairs oppositely to each other in one plane perpendicular to the axis of body 13. Focusing lens 17 is implemented such that the diameter of light spot on the opposite illuminator does not exceed the diameter of this illuminator. Window 19 for photomultiplier 3 is arranged coaxially with the optical system of photomultiplier 3, the axis of which coincides with the axis of body 13.

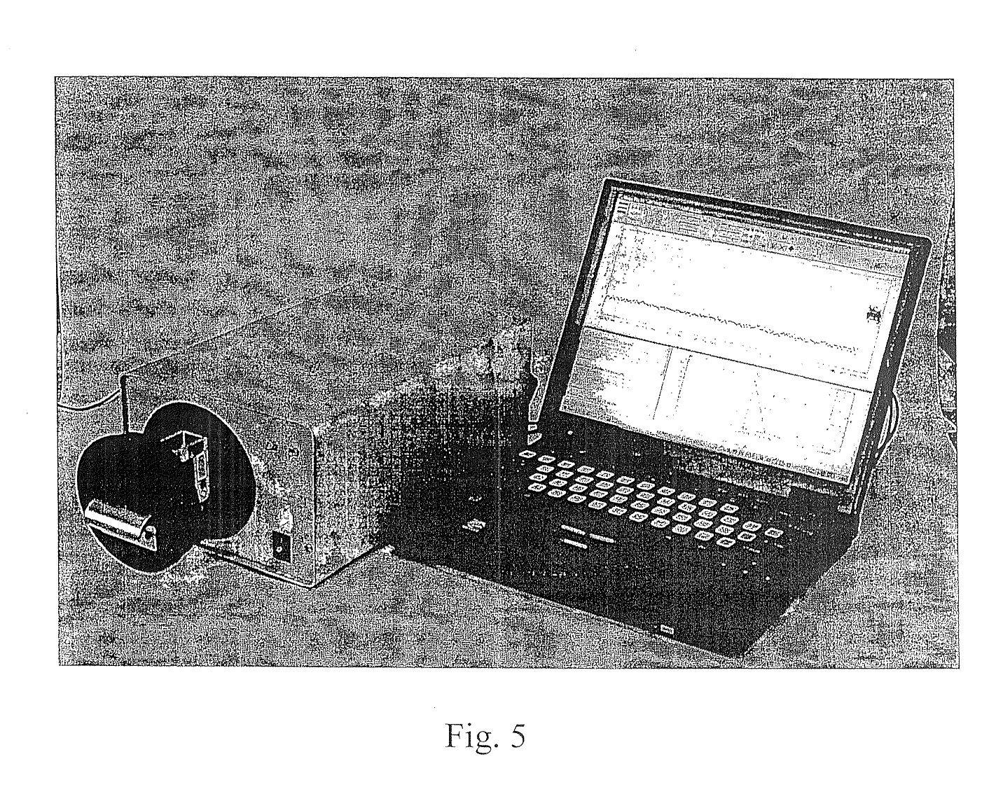

The device units for measuring the first sample piece fluorescence parameters are assembled in a sealed body with an electronic optical measuring system which in combination with the control unit is either an aboard fluorometer shown in FIG. 5, or an immersion probe-fluorometer.

The device is connected to a sampling pump connected to a collector (not shown in the drawing) distributing a sample over two pieces, one of which is fed to measurement chamber 1, and the second one—to the sample concentrating system.

The unit for measuring parameters of chlorophyll fluorescence of individual phytoplankton cells comprises a concentration system, a Nageotte chamber arranged on the table of a LUMAM-IZ-type luminescent microscope with a FMEL 1-A-type fluorometric nozzle of modified with a light-emitting light source, a current stabilizer, a control unit and a computing machine. The described above fluorescence recording module is used for recording and measuring fluorescence of cells.

The device operates as follows.

The pump-selected sample of the studied medium comes into the collector, where the water sample containing phytoplankton is divided into two pieces, one of which is fed to the work volume of measurement chamber 1, and the second one—to the concentration system of the unit for measuring chlorophyll fluorescence parameters of individual phytoplankton cells, after that the second piece is placed in a Nageotte chamber.

Preliminarily, the intensity of natural light acting in the sample harvesting point (natural underwater irradiance) is measured by sensor 8 and fed to control unit 5. From control unit 5 a signal corresponding to the intensity of acting light is fed to light sources 2 through current stabilizer 7 according to the natural irradiance measurements and thereby generating in measurement chamber 1 the illumination corresponding to the natural underwater irradiance.

The intensity of natural light is measured by the number of light quanta per surface unit in a second in the range of 400-700 nm corresponding to the area of photosynthetically active radiation (PAR). Fluorescence emission of algae takes place in a range of waves from 680 nm to 740 nm. Thus, natural light contains an area characteristic of chlorophyll fluorescence not allowing directly, with low fluorescence efficiency (approximately 2%), under effect of natural light measuring the fluorescence intensity with small (characteristic of the natural medium) concentrations of algae. Therefore, in the described device the background illumination simulating natural irradiation is performed in a range of 400-600 nm but equal in the number of quanta to natural irradiation.

Control unit 5 gives in measurement chamber 1 (provided for an opportunity to measure a fluorescence signal) the irradiation intensity equivalent in the number of photons to the conditions of natural irradiation in the sample harvesting point.

A sample piece of phytoplankton placed in measurement chamber 1 wherein the irradiance is created, said irradiance corresponding to the irradiance of the medium in the sample harvesting point and during sample harvesting is exposed to light pulses in accordance with the selected time diagram (FIG. 2).

Due to the even number of light sources and by arranging the light sources by pairs, one opposite other, the optical system of each light source is “a trap” of light for an opposite light source. Such an arrangement of light sources excludes a possibility of multiple scattering on the walls of the measurement chamber decreasing ghost flare light of the fluorescence detector (photomultiplier) with exciting light.

The light arriving from source 2 by means of spherical lens 15 is gathered in a parallel beam which passes through light filter 16 and is focused with long-focus lens 17 in weakly converging beam capable of getting together with the light scattered on a sample in the opposite optical system (FIG. 4).

The light signal of fluorescence a sample through the optical system comprising a light filter emitting the spectral region of chlorophyll fluorescence is recorded with photomultiplier 3. The output signal of photomultiplier 3 comprising information on the size of chlorophyll fluorescence through signal processor 4 is fed to data recording and processing device 6, in this particular case—a personal computer.

The concentration of phytoplankton in the natural medium varies in sufficiently wide ranges from 0.01 μg/l to 100 μg/l. Thus, its concentration and the size of electric signal produced at the output of multiplier 3 are not known beforehand. With the low concentration of phytoplankton an electric signal is small and picked up from amplifier 9 and synchronous detector 10 possessing the maximum amplification coefficient. In increasing the concentration and, accordingly, an electric signal on amplifier 9 signal becomes higher than the maximum value for this amplifier and cannot be objectively measured thereby. Therefore, it is preferred the output signal processor to make as a chain of amplifiers 9-9³ with the known amplification coefficients. Besides, the amplification coefficient of amplifiers 9 is selected to be about 4 in view of that Fm can 4 times exceed Fo allowing the fluorescence signal to be measured per one pulse without adjusting the amplification path. So, with the low concentration of phytoplankton an electric signal is small and is picked up from amplifier 9³ and synchronous detector 10³ possessing the maximum amplification coefficient. In increasing the concentration and, accordingly, an electric signal the amplifier 9 signal becomes higher than the maximum value for this amplifier and cannot be measured thereby. In a cascade circuit a signal is picked up from amplifier 9² with synchronous detector 10², in further increasing fluorescence the signal is picked up from amplifier 9¹ with synchronous detector 10¹ and amplifier 9 with synchronous detector 10. The output signals of synchronous detectors 10 are digitized by analog-digital converter (ADC) 11. Control unit 5 (microprocessor) selects for measuring the fluorescence signal value an unsaturated channel which is closest to the last cascade of amplifiers.

Thus, in one measuring cycle (one pulse) any concentration is measured by selecting an “overswung” amplifier and in view of the relevant amplification coefficient. Such plotting of signal processing allows to enlarge the dynamic range of measurements and do measurements in the initial lighting period at early stages of the induction process and makes it possible to operate in all ranges of phytoplankton concentrations which occur in the natural medium.

The control of measuring cycle is performed by microprocessor control unit 5, from the output of which the signal containing information of chlorophyll fluorescence intensity comes to personal computer 6. In accordance with the findings of measuring the parameters of fluorescence introduced into the built mathematical operation model of the photosynthetic apparatus, the constants of reaction rates of electron transfer indefinable in the indirect experiments are calculated using special programs.

Personal computer 6 in accordance with the selected measuring program gives to control device 5 commands of measuring control, in particular the operation algorithm of current stabilizer 7 and, accordingly, light sources 2, as well as sensor 13 of acting light (irradiance measurer in the sample harvesting point).

In order to evaluate the contribution of individual algal species into the production characteristics of phytoplankton the second sample of the analyzed medium is concentrated (tightened) by means of water filtration through nuclear filters 20. Concentrated algae are placed in a Nageotte chamber having a capacity of 70 μl wherein they are distributed in one layer of cells.

With lower illumination providing visual control of a preparation in the Nageotte chamber, each of the cells being in the field of view is placed by a substage in the photometric zone in diameter of 37.5 μm. By an operator's command, the computer activates a cycle consisting of measuring fluorescence in response to a series of light pulses from the light-emitting diode. By the results of measurements the average values of Fₒ and Fₘ characterizing the abundance of pigments in cells and the efficiency of fluorescent processes by which the distribution of cells of different algal species are judged.

As an example the study of fluorometric characteristics of dominant algae from the coastland of the Black Sea is given.

On the basis of the data of the first study days the dominant species of phytoplankton community were selected and an analysis of distribution of cells was performed by the photosynthesis efficiency value. In the study period the representatives of diatomic Th. nitzsehioides and Rh. fragillissima were depressed. About half these cells had a very low level of photosynthesis efficiency. Using the data obtained on algal cultures and in natural populations, with the level of the average value of (Fₘ−Fₒ)/Fₘ lower than 0.3, the population increase of algal cells discontinues. Therefore, the distribution of cells Th. nitzsehioides and Rh. fragillissima according to the relative variable of fluorescence allows predicting a decline in the population of these species. Most cells of Nitzschia sp. and a representative of dinoflagellates G. wulffii had high photosynthesis efficiency testifying the favorable conditions for them and allowing predicting an increase in the population of these species. Two weeks after the outset of the study the abundance of G. wulffii cells greatly increased. The same but to a somewhat less degree, happened with Nitzschia sp. cells, whereas cells of Th. nitzsehioides and Rh. Fragillissima become to occur episodically.

In table 1 there are given the occurrences of said species of microalgae, the average value of fluorescence intensity Fₒ corresponding to the relative content of photosynthetic pigments of individual cells, and the average value of the relative variable of algal fluorescence (Fₘ−Fₒ)/Fₘ showing the efficiency of the primary photosynthesis processes of organism. Water samples were collected from the “denting” zone (44° latitude 32.8 North and 37° longitude 57.7 East) in September 2002; the content of pigments was calculated by the fluorescence intensity value Fo.

TABLE 1
Species of algae, the contribution of their biomass in the total
biomass of phytoplankton, the content of photosynthetic pigments
and variable of fluorescence (Fm-Fo)/Fm of individual cells.

| | Contribution of | Content of pigments | | | | |
|---|---|---|---|---|---|---|
| | biomass, (Fo), | 10⁻¹² g/cell | | | Fv/Fm, rel. units | |
| Species | % | Min. | Max. | Average | Max. | Average |
| Pseudosolenia calcar-avis | 56 | 18 | 633 | 182 | 0.55 | 0.09 |
| Dactyliosolen fragilissimus | 0.8 | 10 | 95 | 36 | 0.39 | 0.04 |
| Thalassionema nitzschioides | 0.8 | 9 | 67 | 32 | 0.43 | 0.17 |
| Cylindrotheca closterium | 0.01 | 14 | 79 | 38 | 0.63 | 0.28 |
| Pseudo-nitzschia seriata | 1.7 | 17 | 26 | 23 | 0.39 | 0.37 |
| Pseudo-nitzschia delicatissima | 6.0 | 10 | 30 | 17 | 0.23 | 0.04 |
| Chaetoceros affinis | 0.4 | 16 | 491 | 120 | 0.50 | 0.18 |
| Gymnodinium sp. | 0.5 | 9 | 405 | 122 | 0.60 | 0.36 |
| Prorocentrum lima | 1.5 | 145 | 428 | 290 | 0.59 | 0.48 |
| Prorocentrum micans | 3.5 | 171 | 718 | 392 | 0.47 | 0.19 |
| Prorocentrum cordatum | 0.45 | 36 | 100 | 74 | 0.59 | 0.41 |
| Scrippsiella trochoidea | 0.22 | 67 | 251 | 174 | 0.56 | 0.47 |
| Ceratium furca | 3.9 | 60 | 745 | 287 | 0.24 | 0.10 |
| Ceratium fusus | 1.2 | 83 | 637 | 275 | 0 | 0 |

### INDUSTRIAL APPLICABILITY

The inventions will find application in data acquisition for evaluating the functional state of the photosynthetic apparatus of microalgal cells, as well as for short-term forecasts of the population dynamics of some species of phytoplankton in this phytocoenosis. High sensitivity and the operation speed of the described methods allowing reliable measurements to be done for plant objects in the natural conditions, in particular for studying the natural phytoplankton even in very low productive oceanic areas.

The invention is not confined with using for measuring fluorescence of only photosynthesizing organisms, it is also applicable for any cases, in which a series of light flashes allows carrying out a detailed investigation of the processes such as chemical and biological tests based on measuring fluorescence.

1. Method for fluorometrically determining photosynthesis parameters of photoautotropic organisms comprising:
light exposure of a test sample of the analyzed medium to pulses of exciting light with energy sufficient for excitation of chlorophyll fluorescence in the sample followed by measuring the fluorescence intensity according to which the photosynthetic parameters of the investigated object are determined, characterized in that
pulses of exciting light have the same amplitude and variable duration,
wherein the characteristics of chlorophyll fluorescence are measured under constant background illumination simulating the irradiation intensity of an object during studies in the natural conditions and after the adaptation thereof in the dark, and
according to the entirety of fluorescent intensity values the state of a photosynthetic apparatus is determined.

2. Method according to claim 1 characterized in that for determining the chlorophyll fluorescence intensity at which exciting light pulses do not influence the state of the photosynthetic apparatus, the duration of pulses is selected to be 1-5 μs, and an interval between pulses is selected to be 50-100 ms.

3. Method according to claim 1 characterized in that the evaluation of the relative size of a light-harvesting complex of pigments of photosynthetic reaction centers is performed by irradiating an object with a light pulse with duration of 100-200 μs and measuring the chlorophyll fluorescence intensity at least every 10 μs from the outset of the light pulse followed by calculating an increase in the chlorophyll fluorescence intensity while effected by a pulse.

5. Method according to claim 1 characterized in that for evaluating the reduction rate of the components in the acceptor section of photosystem 2 an object is exposed to a series of three groups of light pulses of the same amplitude and duration in each group 1-5 μs, 100-200 μs and 200-1000 ms, respectively, with the average irradiation rate density not less than 3000 J·m⁻²·c⁻¹, the kinetics of changing the fluorescence intensity as affected by each of these pulses separately is measured, wherein the intensity of fluorescence in response to the pulse duration 1-5 μs corresponds to the chlorophyll fluorescence intensity value at which exciting light pulses do not influence the state of the photosynthetic apparatus, by the angle of elevation of the initial section of the induction curve of changing the fluorescence intensity in response to an exciting light pulse with duration of 100-200 μs, the relative size of a light-harvesting complex is determined and by the angle of elevation of the curve of changing the fluorescence intensity in response to the effect of a light pulse with duration of 200-1000 ms the relative size of a quinone pool is determined.

6. Method according to claim 1 characterized in that the maximum level of chlorophyll fluorescence is determined by irradiating the sample with a light pulse with duration of 200-500 ms and the irradiation rate density 3000 J·m⁻²·c^{31 1}.

7. Method according to claim 1 characterized in that for determining the ratio of constants of electron transfer rate in the chain of photosynthetic electron transport the sample is exposed to a light pulse of 300-1000 ms, the chlorophyll fluorescence intensity is measured at least every 1 ms within the duration of said pulse and by the results of the measurements the kinetics of changing fluorescence (induction curves) are obtained.

8. Method according to claim 1 characterized in that a mathematic model of a photosynthetic apparatus is built based on the measurements of the fluorescent parameters, according to said model the quantitative features and constants of electron transfer reactions undetermined experimentally are estimated.

9. Method according to claim 1 characterized in that measurements of the fluorescent parameters are performed on one sample by sequential mode switching of an exciting pulse after measuring each parameter, wherein the average irradiation duration in each next mode is selected higher than in the preceding exposure.

10. Method according to claim 1 characterized in that said method is used to determine photosynthetic characteristics of phytoplankton.

11. Method according to claim 1 characterized in that the selected sample is simultaneously used to determine the contribution of individual species of algae in the production characteristics of phytoplankton, as well as to determine the population heterogeneity of dominant species of algae.

12. Method according to claim 11 characterized in that the second sample of the analyzed medium is isolated from the initially selected sample for determining the contribution of dominant species of algae in the production characteristics, said second sample is concentrated, for example, by tightening by means of water filtration through nuclear filters, the resulting concentrate is distributed in one layer, for example, in a Nageotte chamber, after that the specific composition of cells of phytoplankton organisms is determined by visual evaluation.

13. Method according to claim 11 characterized in that simultaneously with the evaluation of the species of the populations on each cell the fluorescence parameters is measured according to claim 1 and the distribution of different algal species is determined according to the efficiency of the photosynthesis processes and the relative content of pigments in n cell.

14. Device for fluorometrically determining photosynthesis parameters of photoautotropic organisms comprising:
a measurement chamber;
a source of measuring light,
wherein the source of measuring light is optically coupled to the measurement chamber and capable of exciting the fluorescence of a sample;
a module for measuring the sample fluorescence; and
a control unit connected to a data recording and processing unit (a computing machine), the source of measuring light and the module for measuring the sample fluorescence,
characterized in that
said device further comprises:
at least one additional light source optically conjugated to the measurement chamber such that the number of light sources is even;
a current stabilizer of the light sources, wherein the outputs of the current stabilizer are connected to the electrical inputs of the light sources and the input of the current stabilizer is connected to the control unit; and
a sensor of natural irradiation connected to the current stabilizer of the light sources through the control unit.

15. Device according to claim 14 characterized in that said device comprises the same light sources capable of emitting measuring, and/or saturating and/or acting light.

16. Device according to claim 14 characterized in that the module for measuring the sample fluorescence is a fluorescence detector connected to an autonomous high-voltage power supply, for example, a photomultiplier connected to the data recording and processing unit, for example, to a personal computer, through a signal processor and the control unit.

17. Device according to claim 16 characterized in that the signal processor comprises at least one amplifier connected through a synchronous detector to an analog-digital converter the output of which is connected to the control unit.

18. Device according to claim 17 characterized in that the signal processor comprises four series-connected operational amplifiers the output of which each is connected to the analog-digital converter connected to the control unit through the relevant synchronous detector.

19. Device according to claim 14 characterized in that in the preferred embodiment said device comprises a pump, a collector, wherein the first output of the collector is connected to the measurement chamber and the second output of the collector is connected to a concentration system of the second medium sample, an additional measurement chamber for measuring the fluorescent parameters of individual cells, a microfluorometric adapter consisting of a luminescent microscope with a fluorometric nozzle and a light-emitting diode light source connected to the control unit through the current stabilizer of the light sources.

20. Device according to claim 19 characterized in that the fluorometric nozzle may be the module for measuring the sample fluorescence is a fluorescence detector connected to an autonomous high-voltage power supply, for example, a photomultiplier connected to the data recording and processing unit, for example, to a personal computer, through a signal processor and the control unit.

21. Device according to claim 19 characterized in that the additional measurement chamber comprises a Nageotte chamber.

22. Measurement chamber comprising:
a body;
a light source;
a fluorescence detector arranged in the windows of the body; and
inlet and outlet fittings that are capable of feeding to the chamber and removing therefrom a sample of the investigated medium, respectively,
characterized in that
said chamber further comprises:
at least one additional light source arranged diametrically oppositely to the first light source, wherein the additional light source is capable of absorbing light from the first light source.

23. Measurement chamber according to claim 22 characterized in that said chamber comprises the even number of light sources more than two which are arranged by pairs diametrically oppositely to each other in one plane which is perpendicular to the axis of the body, wherein each light source is capable of absorbing light from the oppositely arranged source.

24. Measurement chamber according to claim 22 characterized in that the fluorescence detector is a photomultiplier, the axis of the optical system of the photomultiplier coincides with the axis of the body.
